(19) United States
(12) Patent Application Publication
(10) Pub. No.: US 2010/0286692 A1
(43) Pub. Date: Nov. 11, 2010

(54) EXPANDABLE ORTHOPEDIC DEVICE AND METHOD

(75) Inventors: E. Skott GREENHALGH, Lower Gwynedd, PA (US); John-Paul ROMANO, Chalfont, PA (US); Robert A. KIEFER, Quakertown, PA (US); Wade Kevin TREXLER, Coopersburg, PA (US)

Correspondence Address: LEVINE BAGADE HAN LLP, 2400 GENG ROAD, SUITE 120, PALO ALTO, CA, 94303, US

(73) Assignee: STOUT MEDICAL GROUP, L.P., Perkasie, PA (US)

(21) Appl. No.: 12/839,229

(22) Filed: Jul. 19, 2010

Related U.S. Application Data
(63) Continuation of application No. PCT/US2009/031727, filed on Jan. 22, 2009.
(60) Provisional application No. 61/022,613, filed on Jan. 22, 2008.

(51) Int. Cl. A61B 17/58

(52) U.S. Cl. 606/63

(57) ABSTRACT

A device for radial expansion in an endoluminal cavity in a bone is disclosed. The device can be used to treat bone fractures. The device can have a first radially expandable portion and a second radially expandable portion.

### CROSS-REFERENCE TO RELATED APPLICATIONS

This application is a continuation of PCT Application No. PCT/US09/31727, filed 22 Jan. 2009, which claims priority to U.S. Provisional Application No. 61/022,613, filed 22 Jan. 2008, both of which are incorporated by reference herein in their entireties.

### BACKGROUND OF THE INVENTION

1. Field of the Invention

The present invention relates generally to a device and method for stabilizing bones and anchoring to bones and bone fragments.

2. Description of Related Art

FIG. 1 illustrates a longitudinally split femur bone 8 with the proximal (coronal) end on the right. The femur 10 is a long bone. Bones, such as femurs, have hard, dense cortical outer bone 34, and softer, less dense cancellous inner bone 4 that forms a lumen within the shell of cortical bone 34. FIG. 2 illustrates the endoluminal cavity 6 formed by the cancellous bone 4.

Broken bones, such as long bone breaks, may be treated with fixation. Rigid stabilization rods are often attached to the pedicles of the vertebrae (not shown) with fixation screws. The fixation screws can be driven into the cortical outer shell of the bone.

### SUMMARY OF THE INVENTION

An expandable orthopedic device is disclosed. The device can be a radially expandable attachment device. The device can be used for to therapeutically treat trauma injuries in bones, for example long bone fractures. The device can be fixed in the endoluminal cavity on two sides of a long bone break.

The device can have a structure that can radially expand inside a bone, for example in the endoluminal cavity of a long bone. For example, the device can have a stent-like expandable frame. The device can be implanted in the endoluminal cavity in a radially unexpanded configuration. The device can be radially expanded in the endoluminal cavity using a simple tool, both pump handles, rotational type tools (e.g., cams), or combinations thereof.

The devices can be made from metals, plastics, or combinations thereof, as disclosed infra. The device can be entirely metal, mixes of metal and plastic, entirely plastic, and the device can also have other polymers, agents, fillers and other materials disclosed infra. For example, the radially expandable portion of the device can be a first material (e.g., a first metal) and the remainder of the device can be primarily or entirely made from a second material (e.g., a second metal).

The expansion element can be configured to expand through cancellous bone, and stop when the expansion element contacts hard cortical bone and/or sufficient mechanical resistance. The expansion element can be configured to expand partially or completely into cortical bone, for example to anchor the expansion element into the cortical bone.

The device can be configured to apply a high level of radial force to the inner endoluminal wall of a bone or a low level of radial force.

The device can be designed to stop radially expanding based on displacement (e.g., an internal stop or extent of the length of the radial expansion). The device can be configured to fail mechanically once the device receives a specific mechanical load or resistance, for example for removal or replacement, and/or to prevent the device from over-stressing the bone (e.g., for the device to fail before the bone fails).

The device can contour to the inside of the cortical surface (i.e., outside of the endoluminal cavity) during radial expansion and can anchor to the cortical surface.

Part or all of the outside surface of the device can be textured (e.g., teeth, barbs, hooks, spikes, holes, ridges, knurls, combinations thereof), for example to increase anchoring or improve in-growth of the bone into or towards the device.

The textured surface can be configured to match the required loading. For example, the ridges can be oriented along the longitudinal axis of the device, for example, to resist torque loads on the device by pressing the ridges against the cortical bone accordingly (e.g., in the direction of the torque, producing additional resistance to the torque). The ridges can be oriented perpendicular to the longitudinal axis, for example to resist tensile or compressive loads on the device by pressing against the cortical bone accordingly.

The device can stabilize bone fragments, for example by acting as an endo-scaffold (i.e., a scaffold from within the bone) when in an expanded and/or contracted configuration.

One or more fixation screws can be screwed through bone and into the device—for example when the device is used as an endo-scaffold. The screws can, for example, brace or align the device against or with the bone. By fixing the bone to the device, the bracing element (e.g., fixation screw) can stabilize the bone by pushing the bone radially inward toward the central axis of the bone (e.g., not to one side or the other, for example like a typical external fixation plate or rod). The fracture or bone fragments can be pushed towards the device in the endoluminal cavity. The screws used to screw bone fragments directly onto the device can be lag screws, for example with a distal machine thread.

The device can have any or all elements from any of the devices and/or be used with any method disclosed in U.S. Provisional Application No. 60/906,791, filed 12 Mar. 2007, and PCT App. No. US2008/003421 which are incorporated by reference herein in their entireties. The expandable sections can be made from any configuration (e.g., springs), not just radially expanding bending struts.

The device can be partially or completely hollow. For example, the device can be hollow along the length of the expandable section. The hollow section of the device can be filled with one or more cement, fillers, glues, and/or an agent delivery matrix and/or a therapeutic and/or diagnostic agent. Any of these cements and/or fillers and/or glues can be osteogenic and osteoinductive growth factors.

The device can be implanted anti-grade, retrograde, or constructed from segments from the center of a bone.

The device can be completely or partially bare or covered. The device can have a liner made from a thin film or fabric (e.g., plastic, metal). For example, the liner can control filler flow and/or improve screw anchoring (e.g., by attaching the fixation screw).

The device can be recovered and removed, or repositioned or otherwise adjusted within the endoluminal cavity.

The device can anchor against the cortical bone, for example reducing device motion. The device can be configured to be rigid or flexible.

The device can have one or more radially expandable sections. The expandable sections can be on one or both ends of the implants. The expandable sections can be located along the length of the device at regular or varying length intervals.

The expandable sections can be expanded in any direction (e.g., distal first, proximal last) or out of order along the length of the device. Some expandable sections can be left unexpanded. The expandable sections can be expanded in a sequence to best stabilize the fracture during deployment of the device.

The expandable sections can be used to move the fractured segments of the bone. For example, the expandable section can then be unexpanded and the device moved after the fractured segment of the bone is moved as desired.

The device, for example via the expandable sections, can be used to remove cancellous bone, for example by reaming the endoluminal bone cavity with the expandable section in a radially expanded and/or radially contracted configuration.

A main stem of a joint replacement or resurfacing device can be anchored by having one or more expandable sections in the main stern. The expandable section can be filled with a filler or other material disclosed herein.

The device can be sized and shaped to fit big and small bones (e.g., finger, femurs).

The device can have one or more external guides, for example ridges, rails, threaded holes, or combinations thereof, to guide the screws into the device.

The expandable sections can be expanded by inflating a balloon and/or screw jack (e.g., to bring the longitudinal ends of the expandable section nearer to each other), and/or expanding a wedge-jack inside of the expandable sections.

The fixation screws can have a polyaxial washer head, for example, to distribute stresses. The fixation screws can be linked to one another by a thread, suture, rod, plate, strap or combinations thereof.

The screws can pass through one or more (e.g., two) of the walls of the device. For example, the screw can enter one side and exit the opposite side of the device. A single screw can anchor through cortical bone on substantially opposite sides of the endoluminal cavity.

The fixation screws can have a distal thread. The distal thread can attach into a cell hole of the expandable sections.

The diameter of the screws can be sized to match the diameter of the cell hole.

The expandable section can have one or more layers of walls. The walls can have interconnected struts defining expandable cells. The struts can attach to each other at deformable, resilient and/or rigid joints. Any or all of the remainder of the device can have one or more walls.

The device can be configured so the (longitudinal axis of the) device can be straight and/or curved. The device can be configured to match the topography (i.e., shape) of the endoluminal cavity defined, for example, by the inner wall of the cortical bone. The device can be used to anchor a mesh, suture, or another implant. The device can be curved before radial expansion. The device can be curved and/or bent during radial expansion.

The expandable sections can expand to a round, square, triangular, contoured to the inner wall surface, or combinations thereof cross-sectional configuration. The expandable section can expand into a sphere, rectangle, cube, or contoured any shape to improve anchoring inside a bone.

The device can be used to fill a bone void (e.g., for vertobroplasty (also known as kyphoplasty), tumor therapy, trauma therapy).

Layers of metals, plastic, . . . .

The device can be any length, for example sized to fit a schaphoid or femur.

The device can have expandable sections that can be configured to expand radially, planarly, curvedly, with corresponding wedges, as a polygon, or combinations thereof.

The expandable sections can self-expand (e.g., resilient expansion).

The device can be used with screws, wire, sutures, or combinations thereof. The expandable section can have many holes or few holes.

### SUMMARY OF THE FIGURES

FIGS. 1 and 2 are longitudinal sectional views of a femur.

FIG. 3 illustrates a perspective view of a longitudinally sectioned femur.

FIG. 4 is a sectional view of a femur with the cancellous bone removed.

FIGS. 5 and 6 illustrate a femur.

FIG. 7 illustrates a variation of the device in a contracted configuration at a target site with the bone shown in partial see-through.

FIG. 8 illustrates a variation of the device in an expanded configuration at a target site with the bone shown in partial see-through.

FIGS. 9a through 9c are radiographical images a variation of a method for deploying the device.

FIGS. 10a and 10b are radiographical images of side and end views, respectively, of a variation of the device at a target site in a long bone.

FIGS. 11a and 11b are radiographical images of a variation of the device at a target site in a radially expanded configuration.

FIGS. 12a and 12b are radiographical images of a variation of the device at a target site in a radially expanded configuration.

FIG. 13 illustrates a femur.

FIG. 14 illustrates a method of inserting a variation of the device into a femur.

FIG. 15 is a variation of close-up A-A of FIG. 14.

FIG. 16 illustrates a variation of close-up A-A before the bone is closed (i.e., the fracture is reduced) and stabilized.

FIG. 17 illustrates FIG. 16 with the bone substantially closed and stabilized.

FIG. 18 illustrates a femur.

FIGS. 19 and 20 illustrate a variation of the device at a target site with the bone shown in partial see-through.

FIG. 21 illustrates that the variation of the device at a target site.

FIGS. 22a, 22b and 22c are progressively more magnified close-ups of the distal head of the femur in partial-see though with a device implanted in the femur.

FIG. 23 illustrates an outside view of a variation of the device being inserted and expanded.

FIG. 24 is a radiographical image of a variation of the device in an expanded configuration at a target site.

FIGS. 25a and 25b illustrate progressively more magnified close-ups of the device in a carpel bone with the bone shown in partial see-through.

FIGS. 26a and 26b illustrate progressively more magnified close-ups of the device in a wrist bone with the bone shown in partial see-through.

FIGS. 27a and 27b illustrate variations of transverse cross-sections of the device in unexpanded and expanded configurations, respectively.

FIGS. 28a through 28d are radiographical images of a variation of a method for removing the device from a target site.

FIGS. 29a through 29d are side views of a variation of a method for removing the device and surrounding tissue.

FIGS. 30a through 30d illustrate variations of transverse cross-section C-C of FIG. 10a.

FIGS. 31a and 31b are longitudinal sectional views of variations of the device in an expanded configuration with a locking rod.

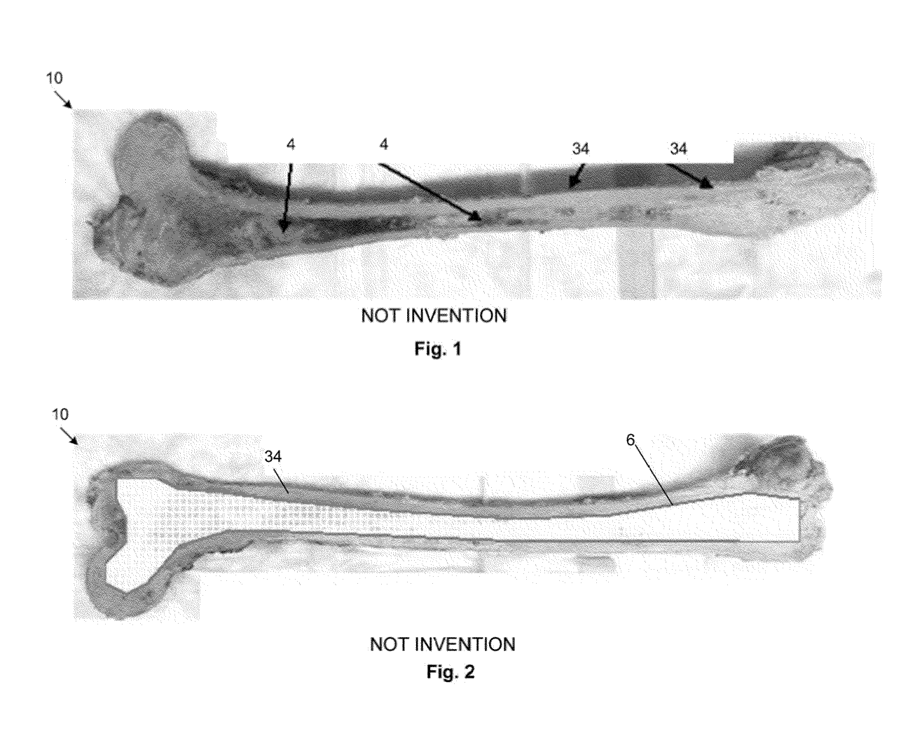

### DETAILED DESCRIPTION OF THE INVENTION

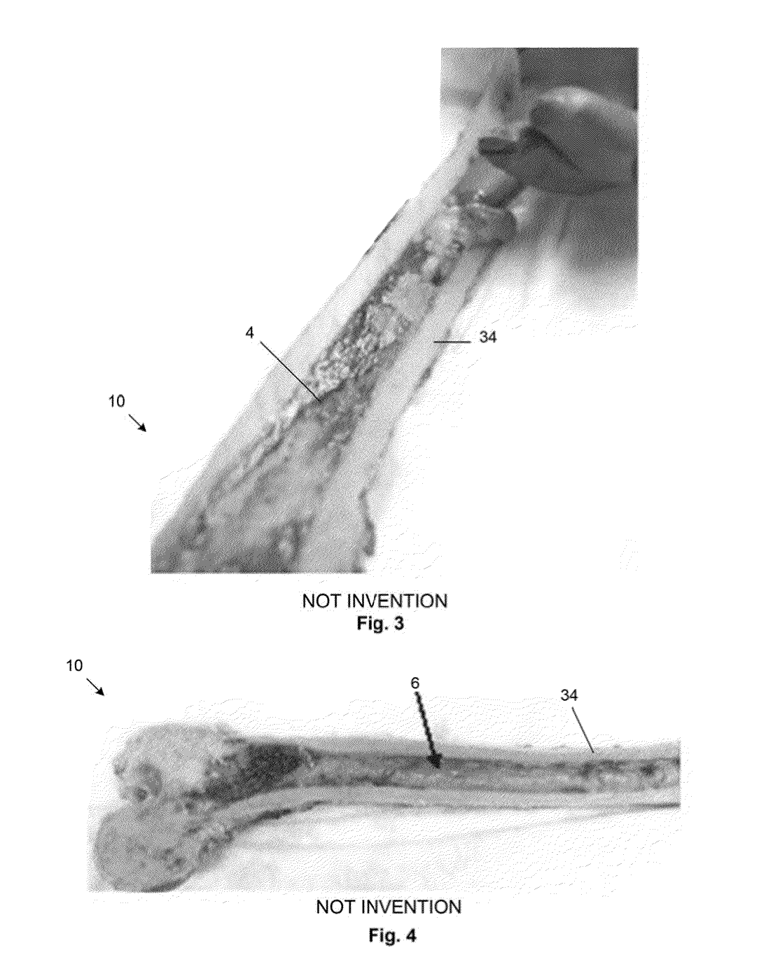

FIG. 3 illustrates that all or part of the cancellous bone 4 can be removed from the endoluminal cavity 6 of a bone 8, such as a long bone 8 such as the femur 10. The cancellous bone 4 can be removed with a finger (as shown), if the cancellous bone 4 is sufficiently soft, or with a reamer or other tool. FIG. 4 illustrates a femur 10 with the cancellous bone 4 removed from the endoluminal cavity 6.

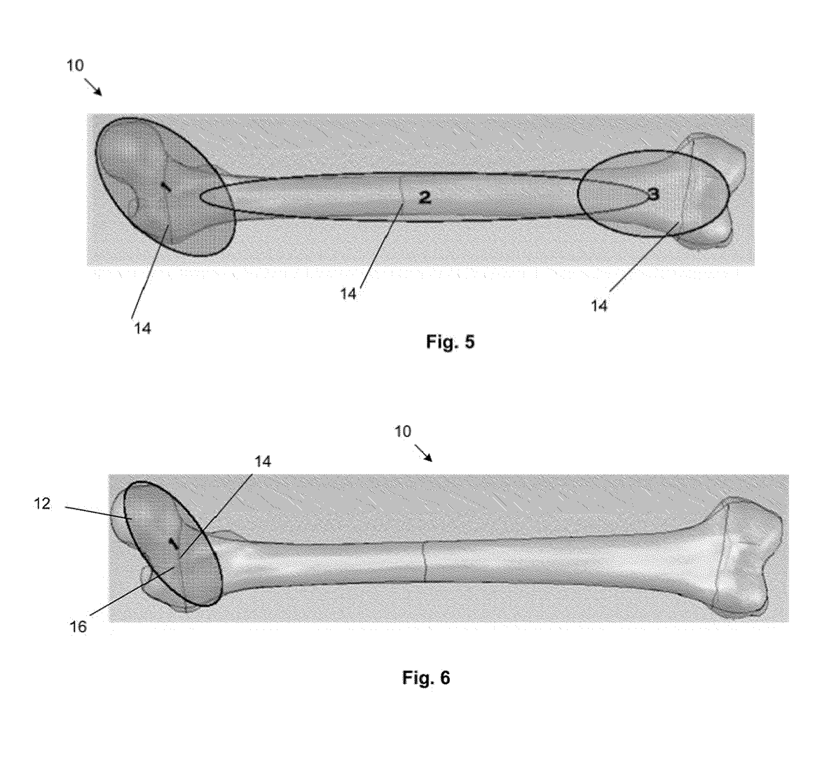

FIG. 5 illustrates three exemplary indications for use of the device include trochanteric fractures 1, mid-shaft fractures 2, distal fractures 3, and combinations thereof. FIG. 6 illustrates the greater trochanter 16 and femoral head 12. The trochanter fracture 14 can bisect the greater trochanter 16.

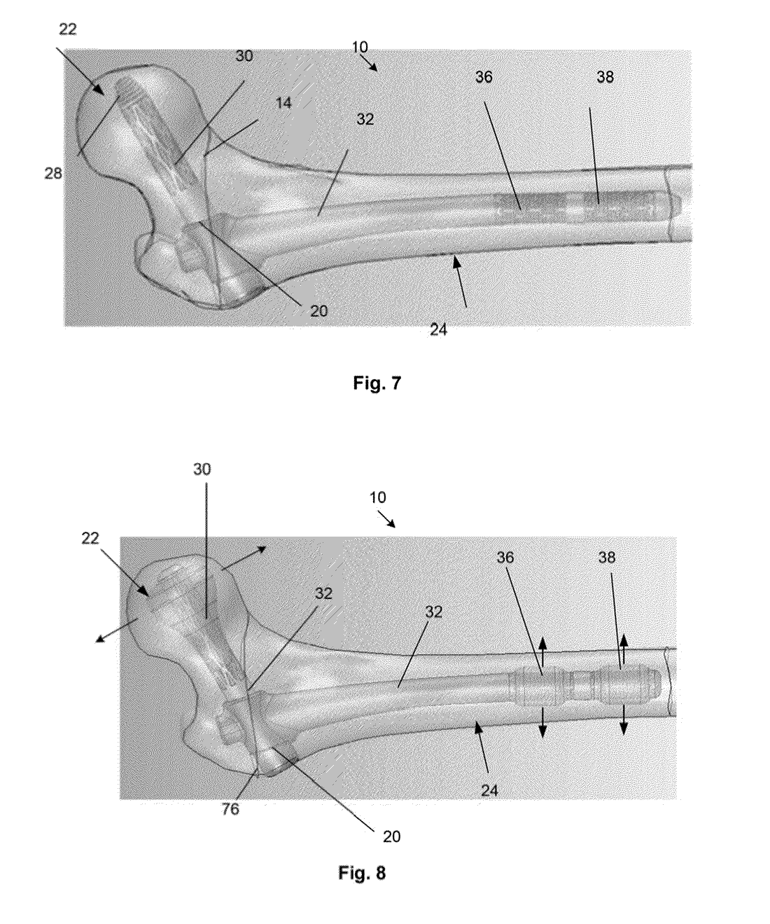

FIG. 7 illustrates that multiple expandable attachment devices 2 can be used in a procedure. For example, a first expandable attachment device 22 can be inserted through the collar 20 of a second expandable attachment device 24 to attach the two devices together.

The first expandable attachment device 22 can be inserted into the femoral head 12. The second expandable attachment device 24 can be inserted along the endoluminal cavity 6 of the femoral shaft.

The first expandable attachment device 22 can have a traumatic screw or otherwise sharpened tip 26. The screw tip 28 can be turned into the bone 8 to help drive and seat the expandable attachment device 2 against the bone 8.

Either or both (shown as just the second) expandable attachment devices 22, 24 can have one or more radial expandable sections 30 and radial unexpandable sections 32. FIG. 8 illustrates that all (as shown) or some of the radial expandable sections 30 can be radially expanded after (or before—not shown) the expandable attachment device 2 is inserted into the bone 8. The expanded expandable sections 30 can secure one or both expandable attachment devices 22, 24 to the inside of the cortical bone 34. At least one of the expandable sections 30 (e.g., the expandable section 30 of the first attachment device 22) can be on a first side of the fracture 14, and at least one of the expandable sections 30 (e.g., both expandable sections 36, 38 of the second attachment device 24) can be on a second side of the fracture 14.

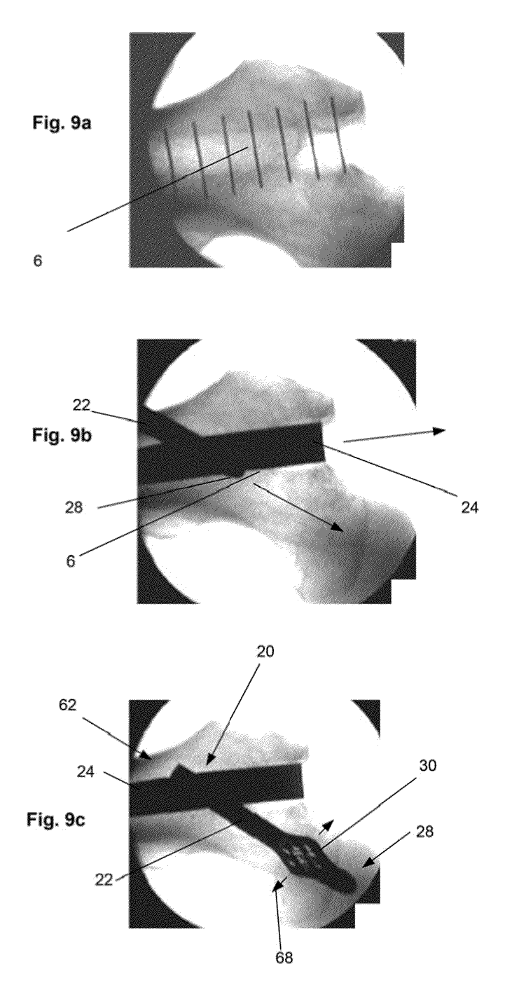

FIG. 9a illustrates that access to the endoluminal cavity 6 can be created by reaming through the cortical bone 34 and remaining out some or all of the cancellous bone 4.

FIG. 9b illustrates that the second expandable attachment device 24 and the first expandable attachment device 22 can be inserted into the bone 8. The first expandable attachment device 22 can be inserted into the bone 8 after the second expandable attachment device 24, for example to insert the first expandable attachment device 22 through the collar 20 (or other attachment element) of the second expandable attachment device 24. Insertion of both expandable attachment devices 22 and 24 is shown by arrows.

FIG. 9c illustrates that the first expandable attachment device 22 can be further inserted through the bone 8, for example by rotating and pushing the first expandable attachment device 22 to utilize the screw tip 28 to drill through the bone 8. The expandable section 30 on the first expandable attachment device 22 (and any other expandable sections 30 desired) can then be radially expanded, as shown by arrows.

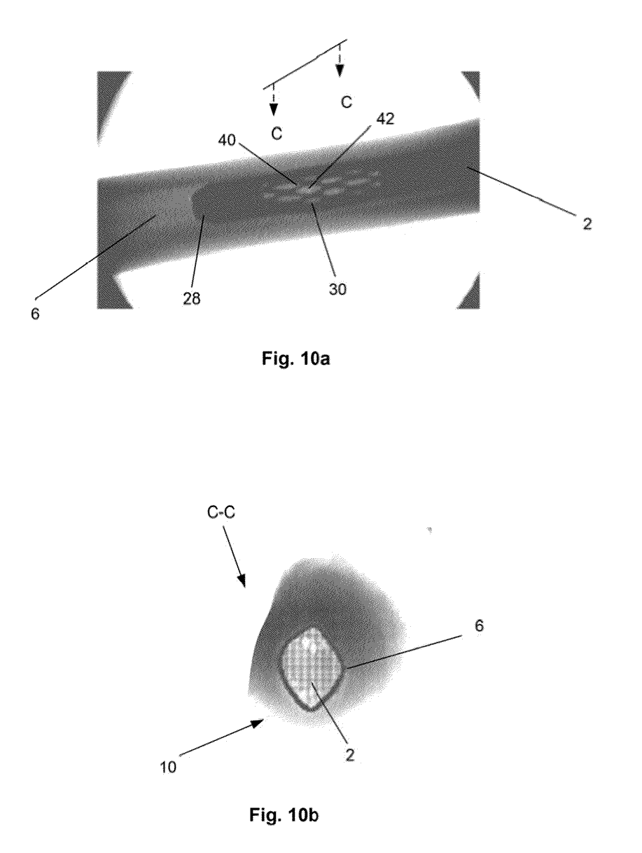

FIG. 10a illustrates a lateral view of the femur 10 with an expandable attachment device 2 inserted into the endoluminal cavity 6 and the expandable section 30 in a radially expanded configuration. The expandable section 30 can conform to the shape of the endoluminal cavity 6 and can secure the expandable section 30 to the cortical bone 34. The expandable section 30 can have struts 40 that can define cells 42 (e.g., openings). The struts 40 can be joined to each other at joints. The struts 40 can be resiliently and/or deformably flexible and/or the joints can be resiliently and/or deformably flexible.

FIG. 10b illustrates an axial view of the femur 10 with the expandable attachment device 2 inserted. FIG. 10b illustrates a variation of transverse cross-section C-C of FIG. 10a.

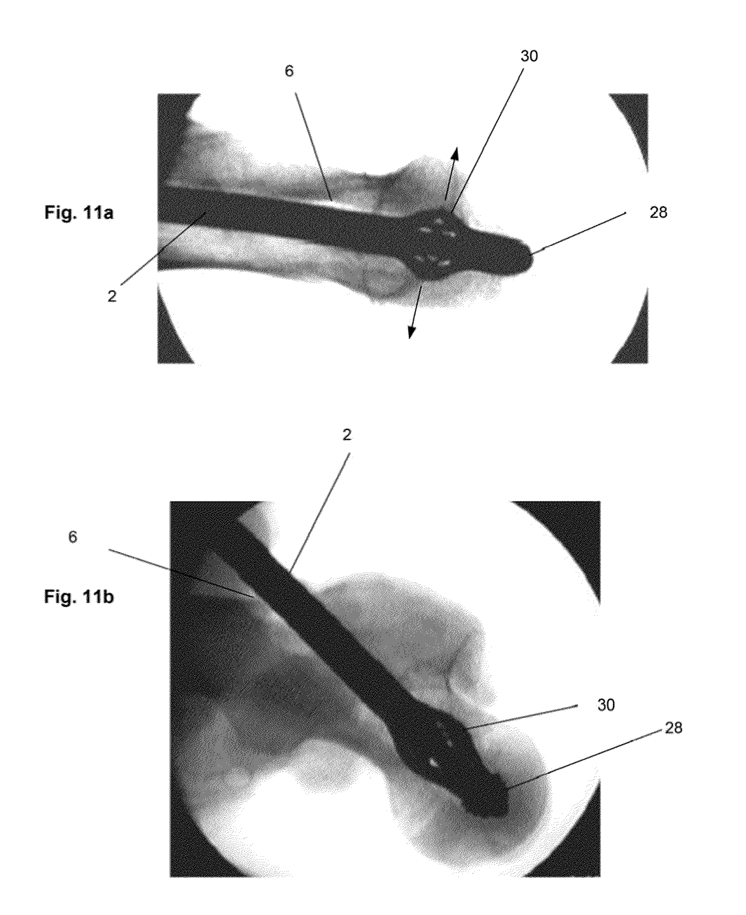

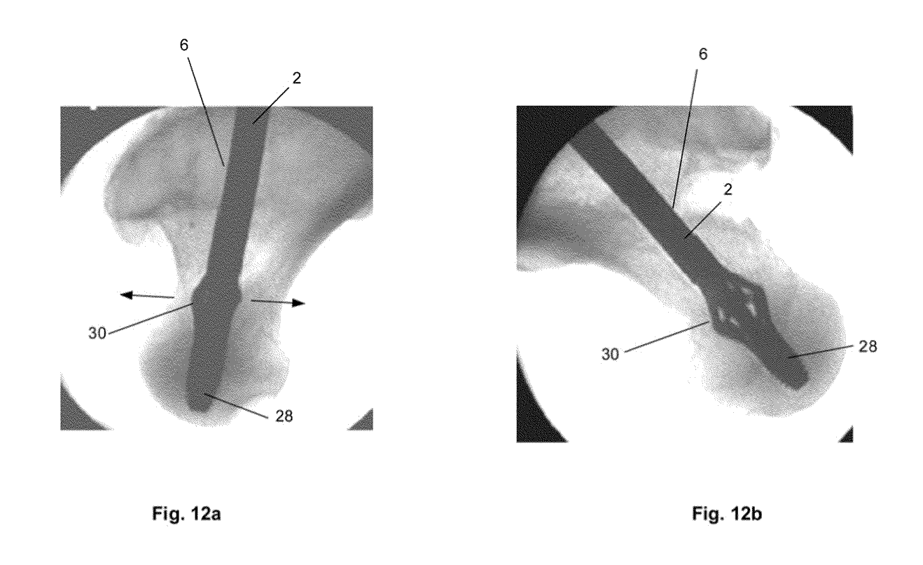

FIGS. 11a and 11b illustrate the expandable attachment device 2 deployed in an osteopenic hip (i.e., in the femur 10). FIGS. 12a and 12b illustrate the expandable attachment device 2 deployed in a healthy hip (i.e., in the femur 10).

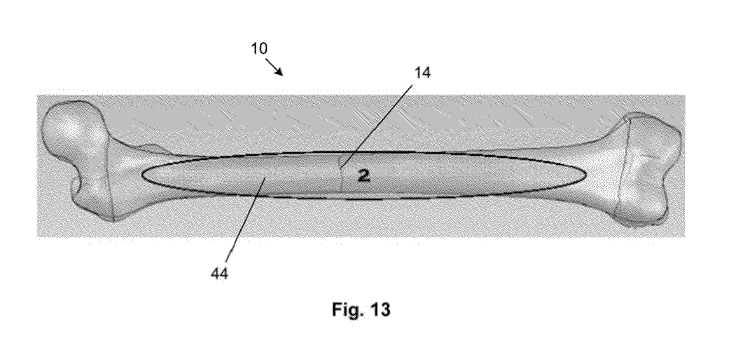

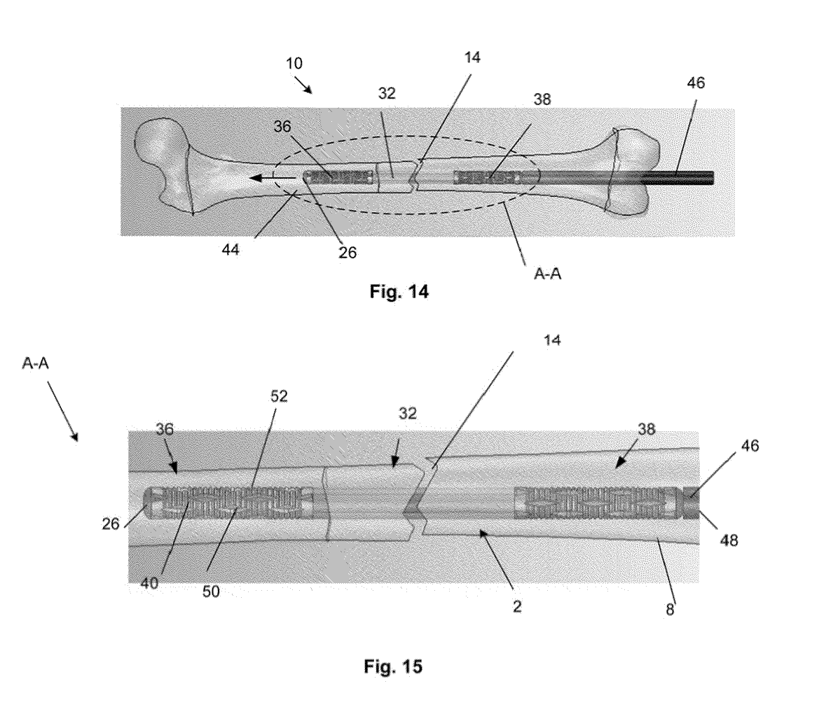

FIG. 13 illustrates that a fracture 14 can be in the femur shaft 44. FIGS. 14 and 15 illustrate that the expandable attachment device 2 can be removably attached to a deployment tool 46. The distal end 48 of the deployment tool 46 can removably attach to the proximal end of the expandable attachment device 2. The deployment tool 46 can position the expandable attachment device 2 (as shown by arrow in FIG. 14). The deployment tool 46 can control radial expansion 68 of each expandable section 30, 32 in unison or independently of one another.

The deployment tool 46, the expandable attachment device 2 or another tool can be used to create a port 50 into the endoluminal cavity 6 and to ream part or all of the endoluminal cavity 6.

The expandable section 30 can have teeth and/or helical threads 52. The teeth or threads 52 can be configured to anchor to the cortical bone 34 when the expandable section 30 is in a radially expanded configuration. The expandable attachment device 2 can have an atraumatic tip 26.

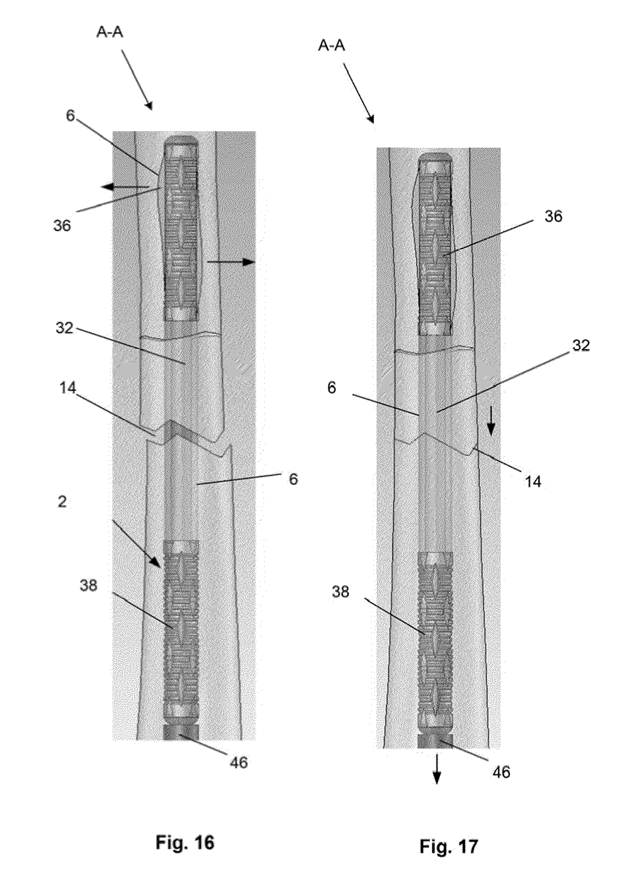

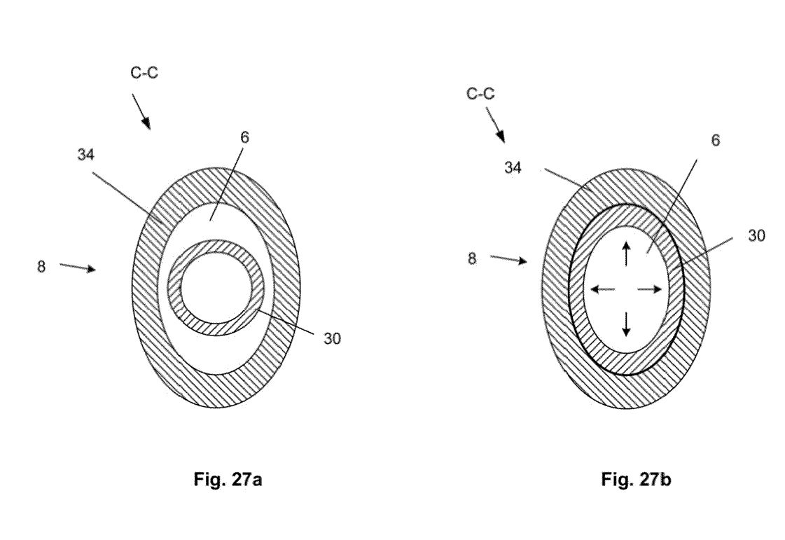

FIG. 16 illustrates that the first expandable section 36, shown at the distal end 48 of the expandable attachment device 2 (which is closer to the proximal end of the femur 10) can be radially expanded first, as shown by arrows. The first expandable section 36 can take on the cross-section of a circle, square, triangle, oval, or otherwise contour to the shape of the endoluminal cavity 6 (as shown—see also FIGS. 27a and 27b), or combinations thereof. The teeth 52 can engage the cortical bone 34 and anchor the expandable section 30 to the cortical bone 34.

FIG. 17 illustrates that the deployment tool 46 can pull, as shown by arrow, the proximal end of the femur 10 toward the distal end 48 of the femur 10 to close the fracture 14. The force to pull the proximal end of the femur 10 toward the distal end 48 of the femur 10 can be transmitted through the unexpandable section 32 and the first expandable section 36 which can be anchored against the cortical bone 34 of the proximal femur 10. The second expandable section 38 can then be radially expanded and the deployment tool 46 can be disconnected and removed from the treatment site (not shown).

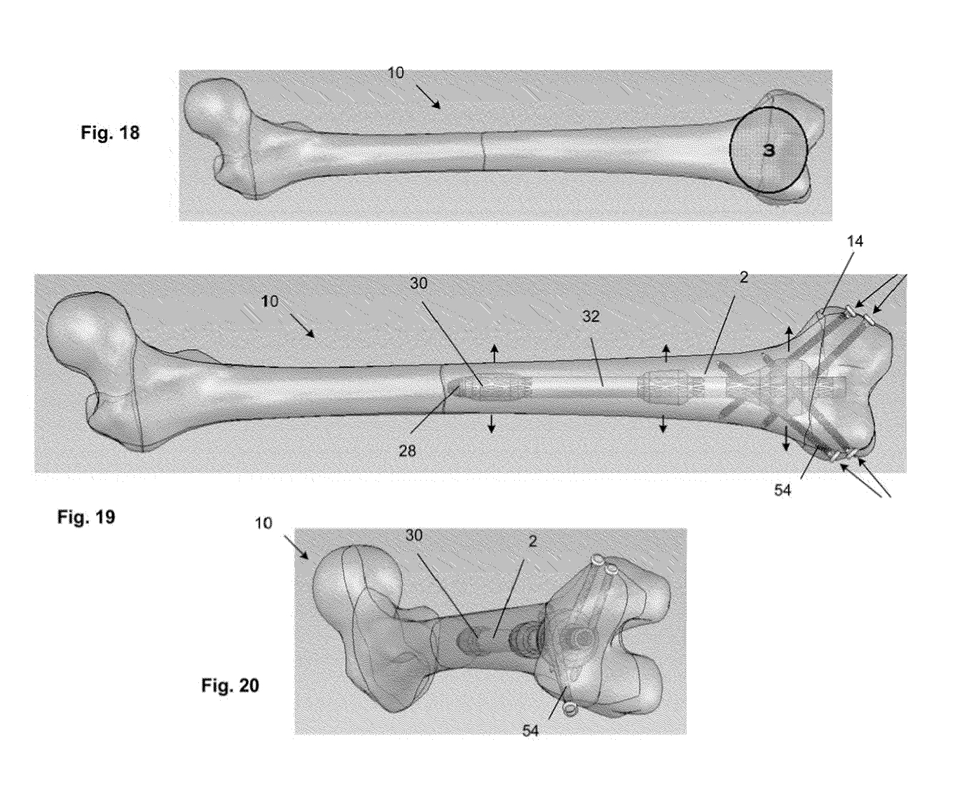

FIG. 18 illustrates a fracture 14 at the distal end 48 of the femur 10. FIGS. 19 and 20 illustrate that the one or more fixation screws 54 can be inserted in one or more directions through the bone 8, and/or across the expandable attachment device 2 and/or the fracture 14. The screws 54 can be inserted through the cells 42 of the expandable section 30. The screws 54 can be sized to be the same size or smaller than the cells 42 when the expandable section 30 is in a radially contracted or radially expanded configuration. The cells 42 can be internally threaded to engage the screws 54. The cells 42, or other holes on the expandable support device can be smaller than, the same size or larger than the screws diameter. For the cells 42 or holes larger than the screws 54, the screws 54 can slide through the hole during and after deployment.

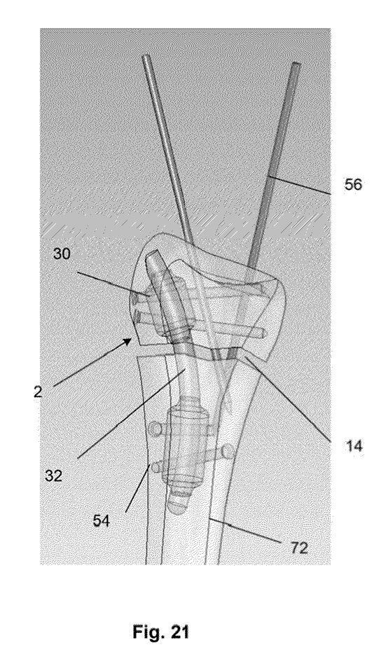

FIG. 21 illustrates that the expandable attachment device 2 can be used for distal radius 72 fracture 14 repair. Additional pins 56 can be placed to secure or control the bone 8 fragments before, and/or during, and/or after deployment of the expandable attachment device 2. The expandable attachment device 2 can be straight or have a substantially non-zero radius of curvature 74. The unexpandable sections 32 and/or the expandable sections 30 can be straight or have a substantially non-zero radius of curvature 74.

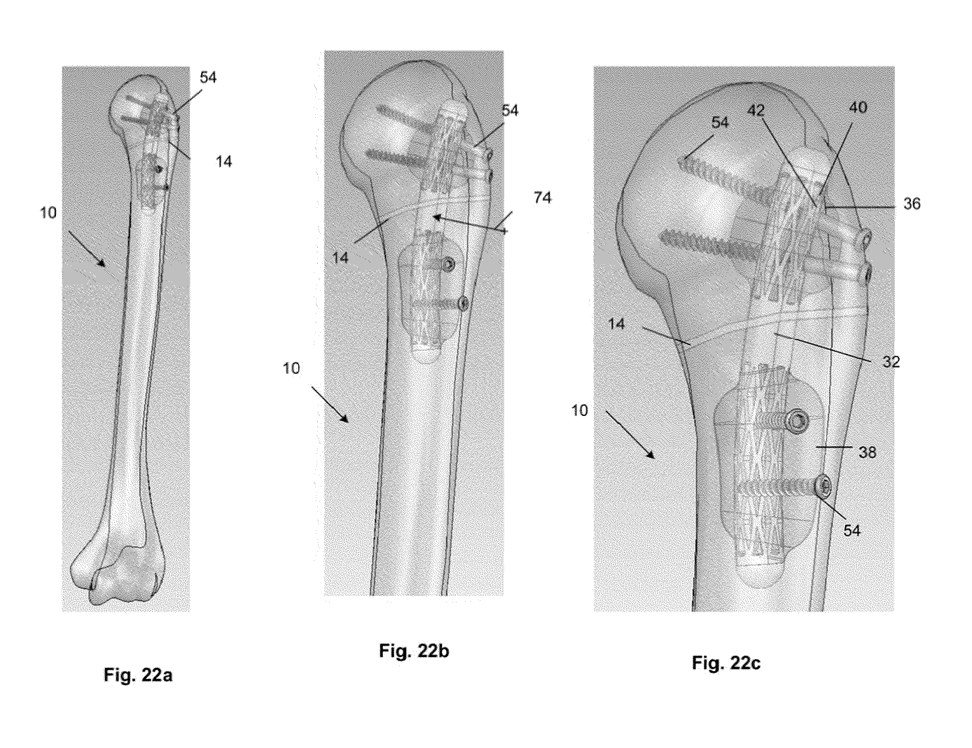

FIGS. 22a through 22c illustrate that the expandable attachment device 2 can be used for proximal humerus fracture 14 repair.

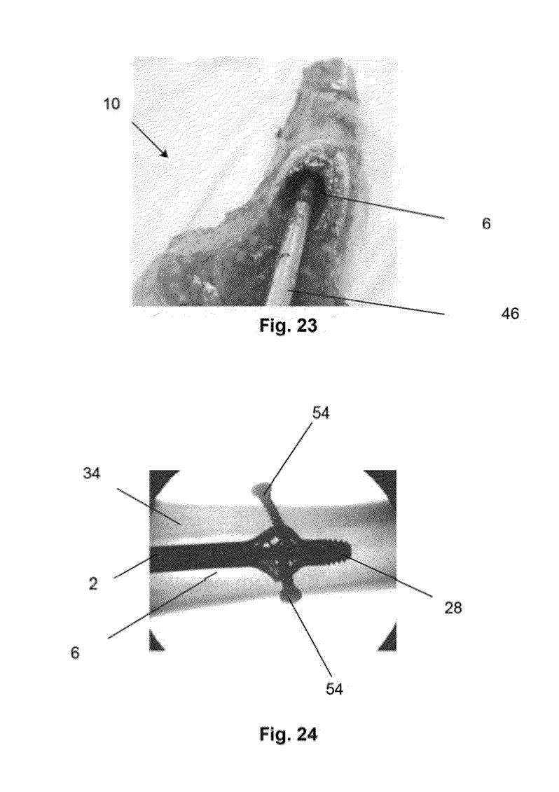

FIGS. 23 and 24 illustrate that the device 2 can be inserted and expanded in an endoluminal cavity 6. FIG. 23 illustrates an external view of the insertion and expansion of the device 2 through the endoluminal channel 6. FIG. 24 illustrates that the bone fixation screws 54 can be inserted through (i.e., nested in) the cells 42 of the expandable section 30. The bone fixation screws 54 can be nested (e.g., pressed against, wedged against) through the cells 42 and/or with the other bone fixation screws 54.

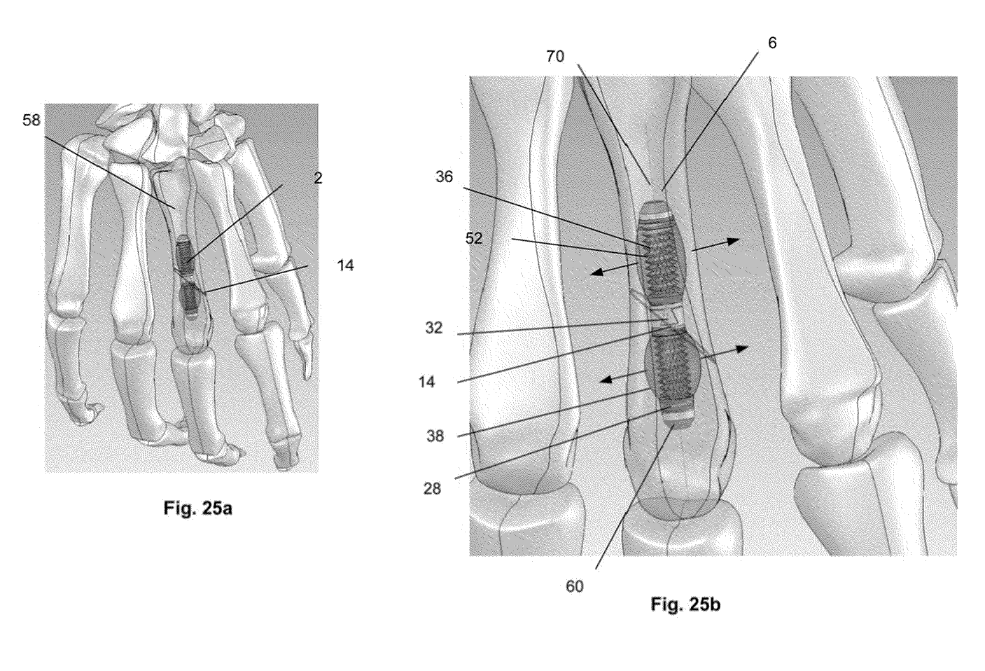

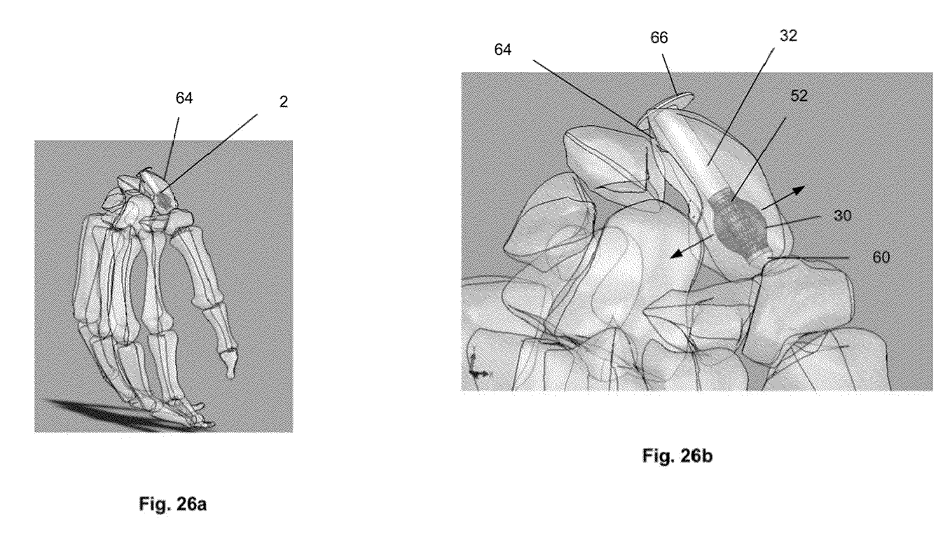

FIGS. 25 and 26 illustrate that the expandable attachment device 2 can be deployed in a phalange 58. For example, the device 2 can be used to treat a broken phalange 58. The expandable attachment device 2 can be modular. The first expandable section 36 can be removed from the second expandable section 38. The unexpandable section 32 can have an interlocking configuration. The physician can construct the expandable attachment device 2 in vivo or in the operating room before or during deployment, for example, to select the best total length of the device to insert, and/or to use combinations of expandable sections 30 with different (or the same) sizes of radial expansion 68.

A traumatic tip 26 (e.g., the screw tip 28) can be covered by an atraumatic tip 26 (e.g., end cap 60) before or after insertion 62 of the device in the treatment site.

FIGS. 26a and 26b illustrate that the expandable attachment device 2 can be deployed in the scaphoid 64. The expandable attachment device 2 can have a proximal anchor 66. The end cap 60 can have a traumatic sharp point, for example, to drive through the bone 8. The proximal anchor 66 can be hit or struck with a hammer or mallet, for example to drive the expandable attachment device 2 into the scaphoid 64 and/or to radially expand the expandable section 30 (e.g., when the resistance against the end cap 60 is greater than the force of the hammer, the expandable section 30 can radially expand).

FIGS. 27a and 27b illustrate that the expandable section 30 of the expandable attachment device 2 can radially expand to fit the shape of the endoluminal cavity 6. FIG. 27a illustrates a transverse cross-section of the bone 8 with the device 2 inserted into the endoluminal cavity 6. FIG. 27b illustrates that the expandable section 30 can radially expand, as shown by arrows, and deform to substantially the same shape as the inner wall of the endoluminal cavity 6. The expandable section 30 can match the interior bone surface contour. For example, this fitting of the expandable section 30 can increase the anchoring force, torque resistance and healing of the bone into the expandable section (i.e., through cells of the stent-like expandable section

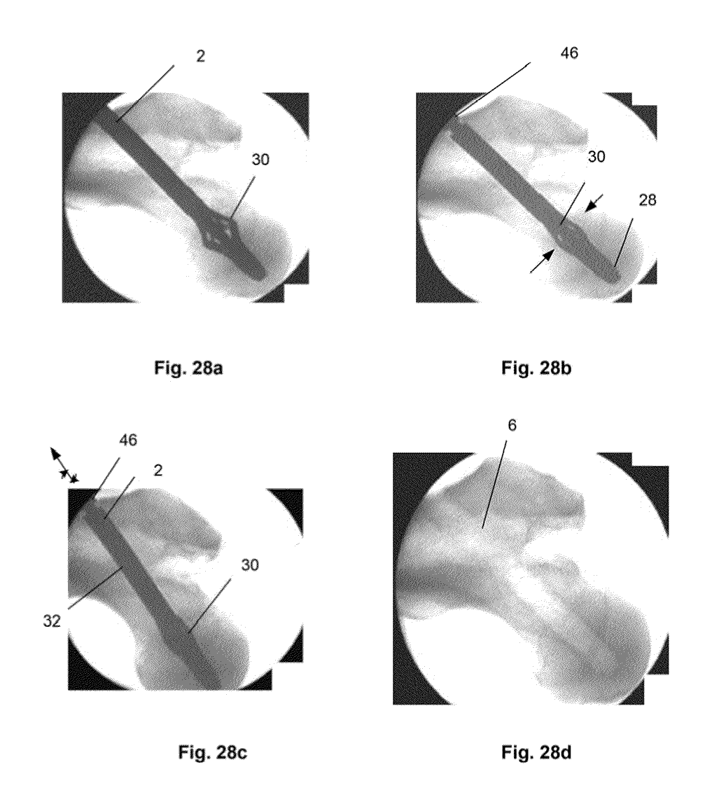

FIGS. 28a through 28d illustrate a sequential method for recovering (i.e., removing) the expandable attachment device 2 from a treatment site. FIG. 28a illustrates that the device 2 can be deployed in a femur, for example extending into the greater trochanter. FIG. 28b illustrates that the expandable section 30 can be radially contracted, as shown by arrows. FIG. 28c illustrates that the device 2 can be unscrewed, or otherwise rotated and/or translated, as shown by arrows, out of the deployment site. FIG. 28d illustrates that the endoluminal cavity 6 can remain in the absence of the device 2. The endoluminal cavity 6 can be completely or partially filled with a material listed herein, such as a bone morphogenic protein or morselized bone.

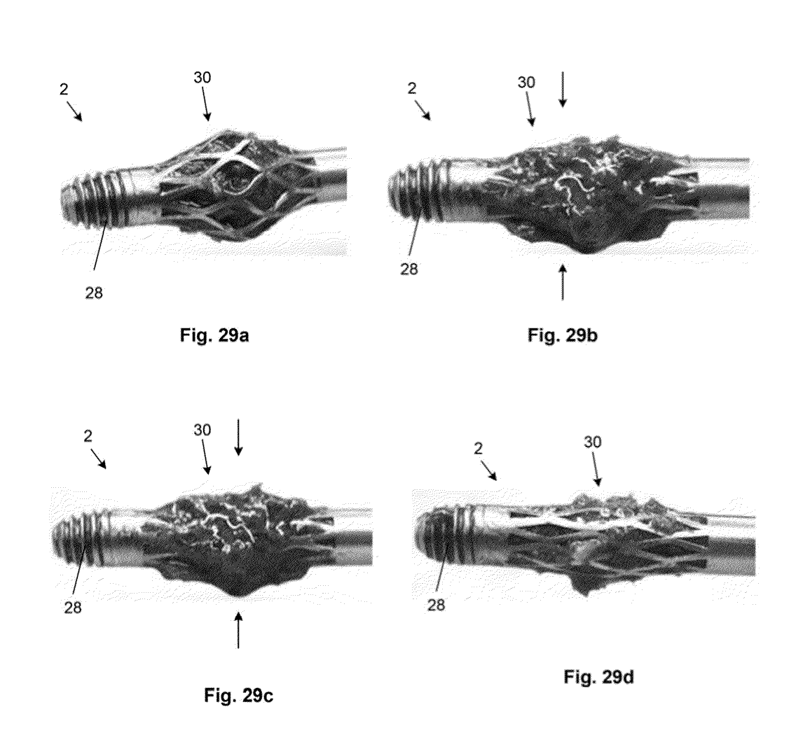

FIGS. 29a through 29d illustrate variations of the distal end 60 of the expandable attachment device 2 during or after removal from or repositioning in a treatment site. The device 2 can be removed from a bone in which the device 2 has been deployed and through which bone has grown.

The expandable section 30, and/or any other hollow section of the device 2 with fluid communication with the outside of the device 2, can be more than about 75% filled, for example about 100% filled with in-grown bone, for example femoral head cancellous bone. The expandable section 30 and hollows of the device 2 can be packed with bone during or after implantation. Merely for example, the deployment site can be in or near a trochanter.

FIG. 29a illustrates that the expandable section 30 of the device 2 can be packed with in-grown bone when implanted in the target site. FIG. 29b illustrates that the expandable section 30 can be partially radially compressed, as shown by arrows. The packed in-grown bone can exit the expandable section 30 via the cells in the stent configuration. FIG. 29c illustrates that the expandable section can be further radially compressed, for example returning the radius of the expandable section 30 to the pre-expansion diameter, and/or an even smaller or a larger diameter than that of the pre-deployment configuration. FIG. 29d illustrates that the expandable support device 2 can be rinsed and/or otherwise cleaned to remove some or all of the bone extending from the cells of the expandable section 30.

The device 2 can be withdrawn from and/or repositioned at the target site at any point during the compression of the expandable section 30 shown in FIGS. 29a through 29d, but more force may be needed to withdraw the device 2 when the expandable section 30 is in an expanded configuration.

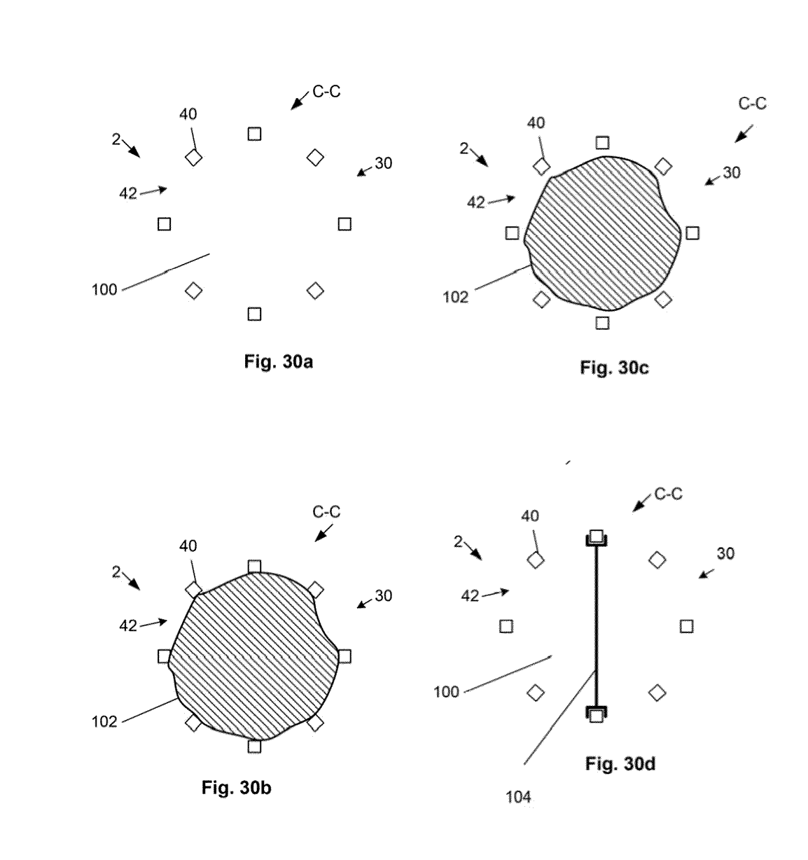

FIG. 30a illustrates that a hollow channel 100 can be defined within the expandable section 30. The hollow channel can be packed, as described herein.

FIG. 30b illustrates that the expandable section 30 can have a bladder or bag 102 attached to the radially inner surfaces of any or all of the struts 40. The bag 102 can be an open, closed (e.g., contained), or closeable structure. The bag 102 (shown filled) can be filled with any material disclosed herein, or combinations thereof, for example morselized bone or bone morphogenic protein. The bag 102 can be porous or non-porous. The bag 102 can be a textile. The bag can cover all or a portion of the expandable section 30. The bag 102 can be partially or completely filled (e.g., by feeding, pumping) before, during or after deployment and expansion of the device 2 in the target site.

FIG. 30c illustrates that the bag 102 can be unattached to the struts 40. For example, the bag can be loose in the hollow channel 100 or the bag can be attached to the device at one or more of the longitudinal ends of the hollow channel 100.

FIG. 30d illustrates that one or more locking rods 104 can be inserted into the expandable section 30 when the expandable section 30 is in an expanded configuration. The locking rods 104 can be oriented transversely, as shown, and/or longitudinally. The locking rod 104 can be resiliently attached to one or more struts and deploy automatically when the expandable section 30 is expanded.

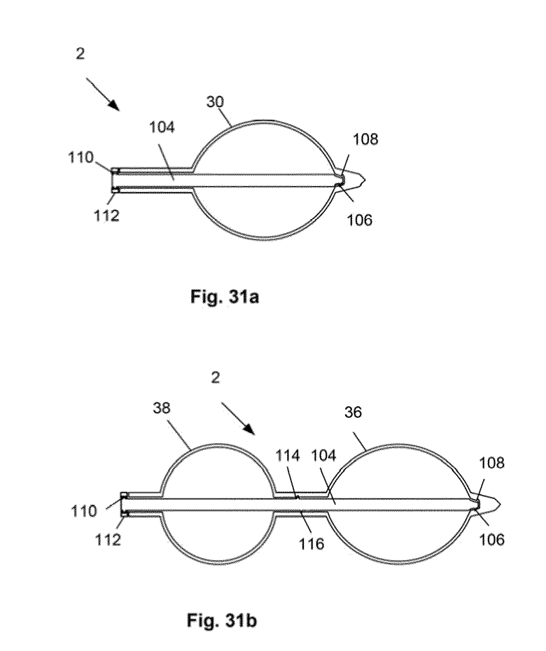

FIG. 31a illustrates that the locking rod 104 can be oriented longitudinally. The locking rod 104 can have distal attachment elements 106, such as one or more threads, ribs, snaps, brads, nuts, clips or combinations thereof, at the distal end of the locking rod. The distal attachment elements 106 can be configured to engageably and releasably attach to one or more distal receiving elements 108 at the distal end of the device 2, such as one or more threads, ribs, snaps, brads, nuts, clips, or combinations thereof.

The locking rod can have proximal attachment elements 110, such as one or more threads, ribs, snaps, brads, nuts, clips or combinations thereof, at the proximal end of the locking rod 104. The proximal attachment elements 110 can be configured to engageably and releasably attach to one or more proximal receiving elements 112 at the proximal end of the device 2, such as one or more threads, ribs, snaps, brads, nuts, clips or combinations thereof.

The locking rod 104 can fix the length between the distal end and the proximal end of the expandable portion 30. The distal attachment elements 104 can engage the distal receiving elements 106 and the proximal attachment elements 108 can engage the proximal receiving elements 110, for example minimizing and/or substantially eliminating the radial compression and longitudinal expansion of the expandable section 30.

FIG. 31b illustrates that the locking rod can have one or more intermediate attachment elements 114 such as one or more threads, ribs, snaps, brads, nuts, clips or combinations thereof. The intermediate attachment elements 114 can be located at a position along the locking rod 104 between the distal attachment elements 106 and the proximal attachment elements 110, for example, such that the intermediate attachment elements 114 can be between the first expandable section 36 and the second expandable section 38 during use. The intermediate attachment elements 114 can be configured to engageably and releasably attach to one or more intermediate receiving elements 116 along the device 2 between the first expandable section 36 and the second expandable section 38. The intermediate receiving elements 116 can be one or more threads, ribs, snaps, brads, nuts, clips or combinations thereof. The intermediate attachment elements 114 can engage the intermediate receiving elements 116, for example, minimizing and/or substantially eliminating the shift of radial expansion to or from the first expandable section 36 from or to, respectively, the second expandable section 38.

Any or all elements of the device and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET), polyester (e.g., DACRON® from E.I. Du Pont de Nemours and Company, Wilmington, Del.), poly ester amide (PEA), polypropylene, aromatic polyesters, such as liquid crystal polymers (e.g., Vectran, from Kuraray Co., Ltd., Tokyo, Japan), ultra high molecular weight polyethylene (i.e., extended chain, high-modulus or high-performance polyethylene) fiber and/or yarn (e.g., SPECTRA® Fiber and SPECTRA® Guard, from Honeywell International, Inc., Morris Township, N.J., or DYNEEMA® from Royal DSM N.V., Heerlen, the Netherlands), polytetrafluoroethylene (PTFE), expanded PTFE (ePTFE), polyether ketone (PEK), polyether ether ketone (PEEK), poly ether ketone ketone (PEKK) (also poly aryl ether ketone ketone), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, osteogenic powder, beads of bone) any of the other materials listed herein or combinations thereof. Examples of radiopaque materials are barium sulfate, zinc oxide, titanium, stainless steel, nickel-titanium alloys, tantalum and gold.

Any or all elements of the device and/or other devices or apparatuses described herein, can be, have, and/or be completely or partially coated with agents and/or a matrix a matrix for cell ingrowth or used with a fabric, for example a covering (not shown) that acts as a matrix for cell ingrowth. The matrix and/or fabric can be, for example, polyester (e.g., DACRON® from E.I. Du Pont de Nemours and Company, Wilmington, Del.), poly ester amide (PEA), polypropylene, PTFE, ePTFE, nylon, extruded collagen, silicone, any other material disclosed herein, or combinations thereof.

The device and/or elements of the device and/or other devices or apparatuses described herein and/or the fabric can be filled, coated, layered and/or otherwise made with and/or from cements, fillers, glues, and/or an agent delivery matrix known to one having ordinary skill in the art and/or a therapeutic and/or diagnostic agent. Any of these cements and/or fillers and/or glues can be osteogenic and osteoinductive growth factors.

Examples of such cements and/or fillers includes bone chips, demineralized bone matrix (DBM), calcium sulfate, coralline hydroxyapatite, biocoral, tricalcium phosphate, calcium phosphate, polymethyl methacrylate (PMMA), biodegradable ceramics, bioactive glasses, hyaluronic acid, lactoferrin, bone morphogenic proteins (BMPs) such as recombinant human bone morphogenetic proteins (rhBMPs), other materials described herein, or combinations thereof.

The agents within these matrices can include any agent disclosed herein or combinations thereof, including radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, SpI Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

Other examples of fractures types that can be treated with the disclosed device and method include Greenstick fractures, transverse fractures, fractures across growth plates, simple fractures, wedge fractures, complex fractures, compound fractures, complete fractures, incomplete fractures, linear fractures, spiral fractures, transverse fractures, oblique fractures, comminuted fractures, impacted fractures, and soft tissue tears, separations (e.g., avulsion fracture), sprains, and combinations thereof. Plastic deformations of bones can also be treated with the disclosed device and method.

Other examples of bones that can be treated with the disclosed device and method include the fingers (e.g., phalanges), hands (e.g., metacarpals, carpus), toes (e.g., tarsals), feet (metatarsals, tarsus), legs (e.g., femur, tibia, fibula), arms (e.g., humerus, radius, ulna), scapula, coccyx, pelvis, clavicle, scapula, patella, sternum, ribs, or combinations thereof. For example, the device can be used in the femoral neck, femoral shaft, proximal or distal tibia or the shaft of the tibia, the humerus, the forearm, the ankle, small bones, the clavicle, and for revision surgery, such as hoip revision surgery.

Any elements described herein as singular can be pluralized (i.e., anything described as “one” can be more than one). Any species element of a genus element can have the characteristics or elements of any other species element of that genus. The above-described configurations, elements or complete assemblies and methods and their elements for carrying out the invention, and variations of aspects of the invention can be combined and modified with each other in any combination.

We claim:

1. A method for repairing a bone fracture comprising:
inserting into an endoluminal channel a device having a first radially expandable portion and a second radially expandable portion, wherein the device has an unexpandable length between the first radially expandable portion and the second radially expandable portion.

2. The method of claim 1, further comprising positioning the first radially expandable portion on a first side of the fracture.

3. The method of claim 2, further comprising positioning the second radially expandable portion on a second side of the fracture.

4. The method of claim 3, further comprising radially expanding the first radially expandable portion.

5. The method of claim 4, further comprising radially expanding the second radially expandable portion.

6. The method of claim 1, further comprising radially expanding the first radially expandable portion.

7. The method of claim 6, further comprising radially expanding the second radially expandable portion.

8. The method of claim 1, further comprising inserting a fixation device through the bone and the first radially expandable portion.

9. The method of claim 5, further comprising inserting a fixation device through the bone and the first radially expandable portion.

10. The method of claim 6, wherein radially expanding the first radially expandable portion comprises deforming the first radially expandable portion to substantially fit an inner contour of the endoluminal channel.

11. The method of claim 6, further comprising radially contracting the first radially expandable portion, and further comprising repositioning the device in the endoluminal channel or removing the device from the endoluminal channel.

12. The method of claim 6, wherein the first radially expandable portion is substantially hollow, and further comprising filling the first radially expandable portion with a filler.

13. The method of claim 12, wherein the filler comprises bone.

14. The method of claim 12, wherein the filler comprises a protein.

15. The method of claim 12, wherein filling comprises filling into a contained bladder.

16. The method of claim 6, further comprising locking the first radially expandable portion in an expanded configuration.

17. A method for repairing a bone fracture comprising:
inserting into an endoluminal channel a device having a first radially expandable portion and a second radially expandable portion.

18. The method of claim 17, further comprising radially expanding the first radially expandable portion and the second radially expandable portion.

19. The method of claim 18, wherein inserting comprises screwing the device into the bone.

20. A method for repairing a bone fracture comprising:
inserting into an endoluminal channel a support element having a first radially expandable portion at a first length along the support element and wherein the expandable portion defines a hollow channel,
radially expanding the first radially expandable portion,
inserting a locking rod into the hollow channel,
attaching the locking rod to the support element distal and proximal to the first radially expandable portion; and
filling the hollow channel with a filler;
wherein the support element has a second radially expandable portion at a second length along the support element, the method further comprising radially expanding the second radially expandable portion, and attaching the locking rod to the support element distal and proximal to the second radially expandable portion.
